(19) United States
(12) Patent Application Publication
(10) Pub. No.: US 2026/0294363 A1
(43) Pub. Date: Oct. 1, 2026

(54) X-RAY DIAGNOSTIC APPARATUS, MEDICAL IMAGE PROCESSING APPARATUS, MEDICAL IMAGE PROCESSING METHOD, AND COMPUTER-READABLE NON-TRANSITORY STORAGE MEDIUM FOR STORING MEDICAL IMAGE PROCESSING PROGRAM

(71) Applicant: CANON MEDICAL SYSTEMS CORPORATION, Otawara-shi (JP)

(72) Inventors: Rumiko YAMAJI, Nasushiobara (JP); Toshio MUROI, Nasushiobara (JP); Nobuo OGURA, Otawara (JP); Yoshiaki IIJIMA, Nasushiobara (JP)

(73) Assignee: CANON MEDICAL SYSTEMS CORPORATION, Otawara-shi (JP)

(21) Appl. No.: 19/553,746

(22) Filed: Mar. 2, 2026

(30) Foreign Application Priority Data
Mar. 31, 2025 (JP) 2025-057804

(51) Int. Cl. A61B 6/12; A61B 6/00; A61B 6/46; G06T 5/50; G06T 5/70; G06T 7/73

(52) U.S. Cl. CPC A61B 6/12; A61B 6/463; A61B 6/469; A61B 6/486; G06T 5/50; G06T 5/70; G06T 7/74; G06T 2207/10016; G06T 2207/10116; G06T 2207/20092; G06T 2207/20224; G06T 2207/30004; G06T 2207/30204

(57) ABSTRACT

An X-ray diagnostic apparatus according to an embodiment includes processing circuitry configured to sequentially acquire a plurality of X-ray images in time series, detect a plurality of candidate target objects as candidates of a plurality of target objects in the X-ray images, calculate evaluation values each for a different candidate target object of the candidate target objects, display the evaluation values each in association with the corresponding candidate target object, and accept a selection of at least one of the plurality of candidate target objects. The circuitry generates a plurality of corrected images by performing correction in which positions of the target objects have been detected in a reference image among the X-ray images and correspond to the candidate target object selected are set as reference positions, and positions of the target objects corresponding to the selected candidate target object are aligned with the reference positions.

### CROSS-REFERENCE TO RELATED APPLICATIONS

This application is based upon and claims the benefit of priority from Japanese Patent Application No. 2025-057804, filed on Mar. 31, 2025, the entire contents of which are incorporated herein by reference.

### FIELD

Embodiments described herein relate generally to an X-ray diagnostic apparatus, a medical image processing apparatus, a medical image processing method, and a computer-readable non-transitory storage medium for storing a medical image processing program.

### BACKGROUND

Endovascular intervention treatment is a treatment method in which a therapeutic instrument, called a catheter, is inserted into a blood vessel to treat lesions that have been developed in organs, such as the heart, brain, and liver. For example, in endovascular intervention treatment, a doctor inserts a balloon catheter into the stenotic site. Then, for example, the doctor injects a liquid into the balloon through the catheter to expand the balloon. As a result, the stenotic site is mechanically dilated, and blood flow is restored. After the liquid in the balloon is aspirated, the balloon catheter is withdrawn from the body by the doctor.

To prevent restenosis of the stenotic site that has been dilated by the balloon, an endovascular intervention treatment is also performed by using a balloon catheter provided with a stent, which is a metallic mesh closely attached to the outer surface of the balloon. In this treatment method, the doctor expands the balloon to deploy the stent, and then aspirates the liquid from the balloon and withdraws the catheter from the body. As a result, the expanded stent remains implanted at the stenotic site, whereby the restenosis rate of the treated stenotic site is reduced.

In endovascular intervention treatment, it is necessary to accurately move the device inserted into a blood vessel to a target treatment site. The positioning of the device is typically performed by referring to X-ray images generated and displayed in real time by an X-ray diagnostic apparatus. For this reason, the device is provided with markers, for example at two positions (or a marker at one position in some cases), made of an X-ray opaque metal, which indicate positions of the balloon and the stent. The doctor positions the device by referring to the markers depicted on the X-ray images displayed on a monitor.

However, when endovascular intervention treatment is performed on blood vessels of organs that constantly pulsate, such as the heart, or organs that move due to pulsation, the position of the device on the X-ray image constantly changes. Therefore, positioning the device by referring to the X-ray image becomes an extremely sophisticated task for the doctor.

Conventionally, a technique is known in which markers depicted in sequentially generated X-ray images are tracked, and each X-ray image is transformed so that respective positions of the markers in the image coincide with its corresponding position in the past image, whereby a video display in which the device appears virtually stationary is provided. Furthermore, as a post-processing technique, it is also known to emphasize the device with high contrast by, for example, performing additive averaging of multiple frames of images in which the marker positions have been corrected to the same location.

### BRIEF DESCRIPTION OF THE DRAWINGS

FIG. 1 is a diagram illustrating an example of a configuration of an X-ray diagnostic apparatus according to an embodiment;

FIGS. 2A and 2B are diagrams illustrating processing that is performed by a detection function according to an embodiment;

FIG. 3 is a diagram illustrating an example of a Learning mode according to an embodiment;

FIG. 4 is a diagram illustrating an example of tracking a plurality of candidate target objects over multiple frames according to an embodiment;

FIGS. 5A and 5B are diagrams illustrating processing that is performed by a corrected image generation function according to an embodiment;

FIG. 6 is a diagram illustrating an example of a Tracking mode according to an embodiment;

FIG. 7 is a diagram illustrating an example of using two different balloons serving as a plurality of target objects according to an embodiment;

FIG. 8 is a table illustrating an example of a track list according to an embodiment;

FIG. 9 is a diagram illustrating an example of a selection screen for markers each corresponding to a different object of candidate target objects according to an embodiment;

FIG. 10 is a diagram illustrating an example of the selection screen displayed on a tablet terminal according to an embodiment;

FIG. 11 is a diagram illustrating an example of another selection screen according to an embodiment; and

FIG. 12 is a flowchart illustrating an example of a procedure of target object selection Tracking processing according to an embodiment.

### DETAILED DESCRIPTION

An X-ray diagnostic apparatus according to an embodiment includes image processing circuitry, processing circuitry, a display, and input circuitry. The image processing circuitry sequentially acquires a plurality of X-ray images in time series. The processing circuitry detects a plurality of candidate target objects as candidates of a plurality of target objects included in the plurality of acquired X-ray images, and calculates evaluation values each for a different candidate target object of the plurality of candidate target objects based on the plurality of X-ray images. The display displays the evaluation values each in association with the corresponding candidate target object of the plurality of candidate target objects. The input circuitry accepts a selection of at least one of the plurality of candidate target objects, The processing circuitry generates a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.

Various embodiments will be described hereinafter with reference to the accompanying drawings.

Hereinafter, embodiments of an X-ray diagnostic apparatus, a medical image processing apparatus, a medical image processing method, and a medical image processing program will be described in detail with reference to the accompanying drawings. Embodiments will be described below centering on an X-ray diagnostic apparatus according to the present specification. A medical image processing apparatus and the X-ray diagnostic apparatus according to the present specification are not limited to the following embodiments. In the following embodiments, elements assigned the same reference numerals perform a similar operation, and duplicated descriptions will be suitably omitted.

### Embodiments

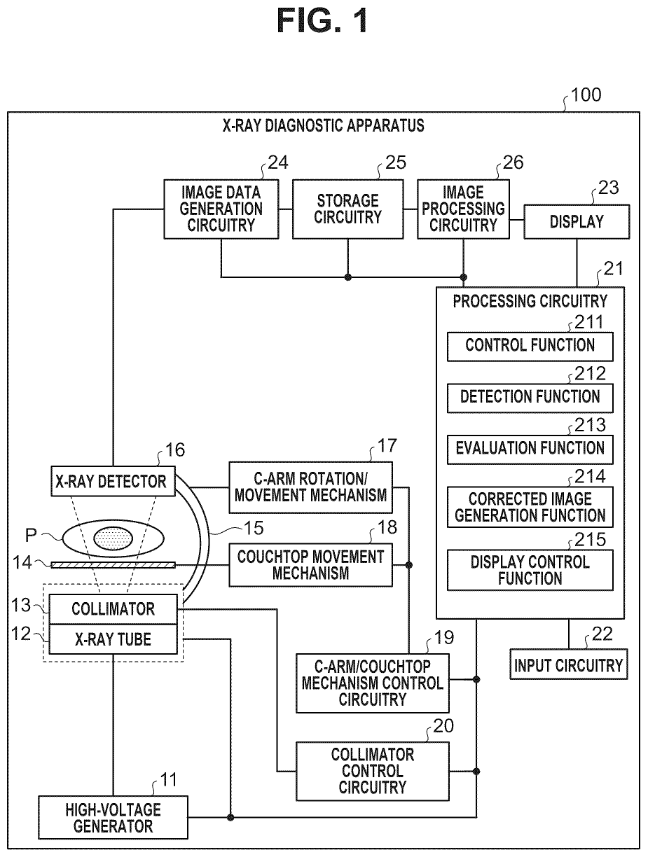

An overall configuration of the X-ray diagnostic apparatus according to an embodiment will be described below. FIG. 1 illustrates an example of a configuration of an X-ray diagnostic apparatus 100 according to an embodiment. As illustrated in FIG. 1, the X-ray diagnostic apparatus 100 according to an embodiment includes a high-voltage generator 11, an X-ray tube 12, a collimator 13, a couchtop 14, a C-arm 15, an X-ray detector 16, a C-arm rotation/movement mechanism 17, a couchtop movement mechanism 18, C-arm/couchtop mechanism control circuitry 19, collimator control circuitry 20, processing circuitry 21, input circuitry 22, a display 23, image data generation circuitry 24, storage circuitry 25, and image processing circuitry 26.

In the X-ray diagnostic apparatus 100 illustrated in FIG. 1, various processing functions are stored in the storage circuitry 25 in the form of computer-executable programs. The C-arm/couchtop mechanism control circuitry 19, the collimator control circuitry 20, the processing circuitry 21, the image data generation circuitry 24, and the image processing circuitry 26 are processors that read the programs from the storage circuitry 25 and execute the programs to implement the functions each corresponding to a different program of the programs. In other words, each circuitry in a state where each program has been read out acquires a function corresponding to the read-out program.

The term “processor” used in the above description refers to circuitry, such as a central processing unit (CPU), a graphics processing unit (GPU), an application specific integrated circuit (ASIC), and a programmable logic device (e.g., a simple programmable logic device [SPLD], a complex programmable logic device [CPLD], or a field programmable gate array [FPGA]).

The processor implements its functions by reading and executing the program stored in the storage circuitry 25. Instead of storing the programs in the storage circuitry 25, the programs may be directly built into the circuitry of the processor. In such a case, the processor implements the functions by reading the built-in program in its own circuitry and executing the program. Each of the processors according to the exemplary embodiments is not limited to a single circuitry element configuration. A plurality of independent circuitry elements may be combined into a processor to implement the function.

Under the control of the processing circuitry 21, the high-voltage generator 11 generates a high voltage and supplies the generated high voltage to the X-ray tube 12. The X-ray tube 12 generates X-rays by using the high voltage supplied from the high-voltage generator 11.

Under the control of the collimator control circuitry 20, the collimator 13 narrows X-rays generated by the X-ray tube 12 so that a region of interest of a subject P is selectively irradiated with the X-rays. For example, the collimator 13 includes four slidable collimator plates. Under the control of the collimator control circuitry 20, the collimator 13 slides these collimator plates to narrow the X-rays generated by the X-ray tube 12 to irradiate the subject P with the X-rays. The couchtop 14 serves as a bed on which the subject P is placed and is disposed on a couch (not illustrated). The subject P is not included in the X-ray diagnostic apparatus 100.

The X-ray detector 16 detects the X-rays that have passed through the subject P. For example, the X-ray detector 16 includes detection elements arranged in a matrix. Each detection element converts the X-rays that have passed through the subject P into an electrical signal, stores the electrical signal, and transmits the stored electrical signal to the image data generation circuitry 24.

The C-arm 15 holds the X-ray tube 12, the collimator 13, and the X-ray detector 16. The X-ray tube 12 and the collimator 13 are disposed to face the X-ray detector 16 by the C-arm 15 with the subject P interposed therebetween. While, in FIG. 1, the X-ray diagnostic apparatus 100 is illustrated as an example of a single-plane type, the embodiment is not limited thereto and may be a bi-plane type system.

The C-arm rotation/movement mechanism 17 is a mechanism for rotating and moving the C-arm 15. The C-arm rotation/movement mechanism 17 can also change the Source Image receptor Distance (SID), which is the distance between the X-ray tube 12 and the X-ray detector 16. The C-arm rotation/movement mechanism 17 can also rotate the X-ray detector 16 held by the C-arm 15. The couchtop movement mechanism 18 is a mechanism for moving the couchtop 14.

Under the control of the processing circuitry 21, the C-arm/couchtop mechanism control circuitry 19 controls the C-arm rotation/movement mechanism 17 and the couchtop movement mechanism 18 to adjust the rotation and movement of the C-arm 15 and the movement of the couchtop 14. Under the control of the processing circuitry 21, the collimator control circuitry 20 adjusts the opening degree of the collimator plates of the collimator 13 to control the irradiation range of the X-rays to be radiated to the subject P.

The image data generation circuitry 24 generates image data using the electrical signal converted from the X-rays by the X-ray detector 16 and stores the generated image data in the storage circuitry 25. For example, the image data generation circuitry 24 performs current-voltage conversion, Analog/Digital (A/D) conversion, and parallel-serial conversion on the electrical signals received from the X-ray detector 16 to generate image data (projection data). The image data generation circuitry 24 stores the generated image data in the storage circuitry 25.

The storage circuitry 25 receives and stores the image data generated by the image data generation circuitry 24. In addition, the storage circuitry 25 stores the programs each corresponding to a different function of various functions that are read and executed by the respective circuitry illustrated in FIG. 1. For example, the storage circuitry 25 stores a program corresponding to a control function 211, a program corresponding to a detection function 212, a program corresponding to an evaluation function 213, a program corresponding to a corrected image generation function 214, and a program corresponding to a display control function 215, which are read and executed by the processing circuitry 21.

Under the control of the processing circuitry 21 described below, the image processing circuitry 26 performs various image processing on the image data stored in the storage circuitry 25 to generate X-ray images. Alternatively, under the control of the processing circuitry 21 described below, the image processing circuitry 26 directly acquires image data from the image data generation circuitry 24 and performs various image processing on the acquired image data to generate X-ray images. The image processing circuitry 26 can also store X-ray images after the image processing in the storage circuitry 25. For example, the image processing circuitry 26 can perform various processing by using image processing filters, such as a moving average (smoothing) filter, a Gaussian filter, a median filter, a recursive filter, and a band-pass filter.

The input circuitry 22 is implemented by devices, such as a track ball, switch buttons, a mouse, and a keyboard, for specifying a region (e.g., region of interest), and devices, such as a foot switch, for performing X-ray irradiation. The input circuitry 22 is connected to the processing circuitry 21 and converts an input operation received from an operator into an electrical signal, and outputs the signal to the processing circuitry 21. The input circuitry 22 accepts various operations performed by the operator. The input circuitry 22 may be referred to as an input unit, an operation unit, an acceptance unit, or the like.

The display 23 provides Graphical User Interfaces (GUIs) for receiving operator instructions and displays various images generated by the image processing circuitry 26. Examples of displays that can be used as the display 23 include a Liquid Crystal Display (LCD), a Cathode Ray Tube (CRT) display, an Organic Light-Emitting Diode (OELD) Display, a plasma display, and other optional displays. The display 23 may be a desktop type, or a tablet terminal and the like capable of wireless communication with the processing circuitry 21. The display 23 corresponds to a display unit.

The processing circuitry 21 controls overall operation of the X-ray diagnostic apparatus 100. More specifically, the processing circuitry 21 reads the program corresponding to the control function 211 for controlling the entire apparatus from the storage circuitry 25 and executes the program to perform various processing. The processing circuitry 21 may also be referred to as a processing unit.

For example, the processing circuitry 21 controls, by the control function 211, the high-voltage generator 11 according to an operator instruction transferred from the input circuitry 22 to adjust the voltage to be supplied to the X-ray tube 12, whereby the X-ray dose to be irradiated to the subject P and the ON/OFF state of the X-ray radiation are controlled. For example, the control function 211 controls the C-arm/couchtop mechanism control circuitry 19 according to an operator instruction to adjust the rotation and movement of the C-arm 15 and the movement of the couchtop 14. Furthermore, for example, the control function 211 controls the collimator control circuitry 20 according to an operator instruction to adjust the opening degree of the collimator plates of the collimator 13, whereby the irradiation range of the X-rays to the subject P is controlled.

The control function 211 controls the image data generation processing by the image data generation circuitry 24, the image processing or analysis processing by the image processing circuitry 26, and the like in accordance with operator instructions. Further, the control function 211 controls the display 23 to display a GUI for receiving operator instructions and images stored in the storage circuitry 25. The processing circuitry 21 for implementing the control function 211 may also be referred to as a control unit.

As illustrated in FIG. 1, the processing circuitry 21 executes the detection function 212, the evaluation function 213, the corrected image generation function 214, and the display control function 215. Processing implemented by the detection function 212, the evaluation function 213, the corrected image generation function 214, and the display control function 215 will be described in detail below.

The image processing circuitry 26 and/or the image data generation circuitry 24 described above is an example of an acquisition unit that sequentially acquires a plurality of X-ray images in time series. The detection function 212 is an example of a detection unit. The evaluation function 213 is an example of an evaluation unit. The corrected image generation function 214 is an example of a corrected image generation unit. The display control function 215 is an example of a display control unit.

As described above, the overall configuration of the X-ray diagnostic apparatus 100 has been explained. The X-ray diagnostic apparatus 100 according to the present embodiment is capable of selecting a plurality of target objects included in the X-ray images and improving the visibility of the selected target object. For example, the X-ray diagnostic apparatus 100 according to the present embodiment enables the selected target object to be fixed for display on the X-ray images. More specifically, the X-ray diagnostic apparatus 100 enables the display of a moving image in which the target object corresponding to a candidate target object selected by the operator (a treatment instrument or device) appears virtually stationary. The target object is, for example, an instrument inserted into the body of the subject P. Examples of the instrument inserted into the body of the subject P include a stent, a marker (feature object), a wire, and other treatment devices. In other words, the target object is at least one of a treatment device inserted into the body of the subject P and a feature object attached to the treatment device (such as a marker pair or a pair of markers to be described below). The treatment device may include a plurality of feature objects.

For example, when a doctor performs endovascular intervention treatment on a stenotic site in a cardiac vessel of the subject P using a “balloon catheter with a stent”, the doctor positions the device by referring to the X-ray images generated and displayed by the X-ray diagnostic apparatus 100. As described above, when endovascular intervention treatment is performed on blood vessels of organs that constantly pulsate, such as the heart, or organs that move by pulsation, the position of the device on the X-ray images changes, making it extremely difficult for the doctor to position the device by referring to the X-ray image.

For this reason, the X-ray diagnostic apparatus 100 tracks markers at multiple points depicted in sequentially generated X-ray images, and performs image correction so that the positions of the pair of markers in each X-ray image coincide with their positions in a past image, thus a moving image in which the device appears to be virtually stationary is displayed. For example, the X-ray tube 12 irradiates the region of interest (e.g., the heart) of the subject P with X-rays, and the X-ray detector 16 sequentially detects the X-rays that have passed through the region of interest. Based on data continuously detected by the X-ray detector 16, the X-ray diagnostic apparatus 100 performs image processing so that the device included in the X-ray images sequentially generated in time series appears virtually stationary, and displays the processed images as a moving image in real time.

This enables the X-ray diagnostic apparatus 100 to display X-ray images with improved visibility of the device displayed during endovascular intervention treatment performed by referring to X-ray images, whereby device positioning is facilitated. However, in the above-described technique, it is sometimes not possible to perform image processing so that a desired device appears virtually stationary and display the processed images as a moving image in real time.

Thus, the X-ray diagnostic apparatus 100 according to the present specification, through the processing circuitry 21 described in detail below, performs image processing so that a device appears virtually stationary with respect to a pair of markers including a marker selected from among markers at multiple points by the user and another marker associated with the selected marker, and displays the processed image as a moving image in real time. The X-ray diagnostic apparatus 100 according to the present specification allows selection of a plurality of target objects included in the X-ray images and improves the visibility of the selected target object.

Processing for displaying a moving image in which the device appears to be virtually stationary will be described below. This processing includes, for example, executing image processing on the collected X-ray images to detect a plurality of marker candidates. Then, the processing identifies a plurality of pairs of markers from among the plurality of marker candidates, and tracks the plurality of identified pairs across multiple frames. This processing enables selection of the most plausible stent marker pair. Subsequently, the processing calculates a shift amount so that the selected pair of markers is displayed at the center of the screen and displays the image centered on the stent markers.

The following describes a case in which the processing circuitry 21 executes various functions and controls the image processing circuitry 26 to perform processing. However, the processing circuitry 21 may alternatively execute similar processing to that of the image processing circuitry 26.

More specifically, when displaying a moving image in which the device appears virtually stationary, the detection function 212 controls the image processing circuitry 26 to use a group of image data sequentially generated by the image data generation circuitry 24 during a predetermined period to identify a plurality of candidate target objects as a plurality of candidates of target objects related to the medical device inserted into the body of the subject P, and detect the positions of the plurality of candidate target objects in a newly generated X-ray image based on a result of the identification. More specifically, the detection function 212 controls the image processing circuitry 26 to detect a plurality of target objects included in X-ray images generated from the image data. The predetermined period for the target object detection and the predetermined detection target object to be detected may be determined at any time before the detection processing starts, for example, before generation of the image data group, during generation of the image data group, or after generation of the image data group.

For example, each time a new image (new X-ray image) is stored, the detection function 212 detects the coordinates of the stent markers attached to the stent in the new image. More specifically, based on information about the stent markers depicted in the image, the detection function 212 detects the coordinates of the stent markers in the sequentially generated X-ray images. For example, the detection function 212 detects the coordinates of the stent markers in the sequentially generated X-ray images based on information about the stent markers specified by the operator or a training image of the stent markers.

More specifically, the detection function 212 generates frequency images including predetermined frequency components from the sequentially generated X-ray images and detects the coordinates of a plurality of candidate target objects included in the plurality of generated frequency images. More specifically, the detection function 212 generates high-frequency images including high-frequency components from the sequentially generated X-ray images, and detects the coordinates of the stent markers in the generated high-frequency images. More specifically, the predetermined frequency components are frequency components containing components corresponding to a plurality of target objects. The detection function 212 generates frequency images in which predetermined target objects are emphasized and detects coordinates of the predetermined target objects. The predetermined frequency components in the frequency images may be determined at any time before the detection processing starts, for example, before generation of the image data group, during generation of the image data group, or after generation of the image data group.

The detection function 212 also generates low-frequency images of X-ray images by performing smoothing processing on the X-ray images. The detection function 212 detects the coordinates of the stent markers in the generated high-frequency images. For example, the detection function 212 performs the above-described processing on the sequentially generated X-ray images to generate a high-frequency image for each X-ray image and detect coordinates of the stent markers included in the generated high-frequency images.

More specifically, the detection function 212 generates high-frequency images, i.e., X-ray images with low-frequency components eliminated, by subtracting low-frequency images from X-ray images. Then, the detection function 212 performs binarization on the high-frequency images. Subsequently, the detection function 212 removes wire images (connected regions having a large area) to detect coordinates of the plurality of candidate target objects. In this processing, the detection function 212 calculates the reliability (Trustness) of the plurality of candidate target objects based on templates of the plurality of candidate target objects and pixel values in the coordinates of the plurality of candidate target objects. More specifically, the detection function 212 calculates the marker-likeness corresponding to the reliability of a plurality of candidate target objects based on the marker templates and pixel values of a plurality of markers. The marker-likeness can be calculated by using a known technique, and descriptions thereof will be omitted.

In this processing, the detection function 212 compares the marker-likeness with a threshold value stored in the storage circuitry 25 to identify (select) coordinates of markers whose marker-likeness exceeds the threshold value. The threshold value is preset and stored in the storage circuitry 25. The calculation of the marker-likeness may be performed by the evaluation function 213. The selected coordinates correspond to the marker-likeness exceeding the threshold value. The coordinates identified (selected) by the detection function 212 correspond to the coordinates of the plurality of candidate target objects.

For example, the detection function 212 selects pair candidates (a plurality of candidate target objects) that fall within a preset range of the stent length. Then, the detection function 212 tracks the selected pair between consecutive X-ray images in time series (adjacent frames) in a plurality of X-ray images by using the center coordinates of the selected pairs. The generation of high-frequency images is not limited to the above-described example. For example, the generation may be performed by any desired method, such as processing using a band-pass filter.

Processing for displaying a moving image in which the device appears virtually stationary will be described below centering on an example in which coordinates of two stent markers are detected. Processing after the generation of high-frequency images will be described below.

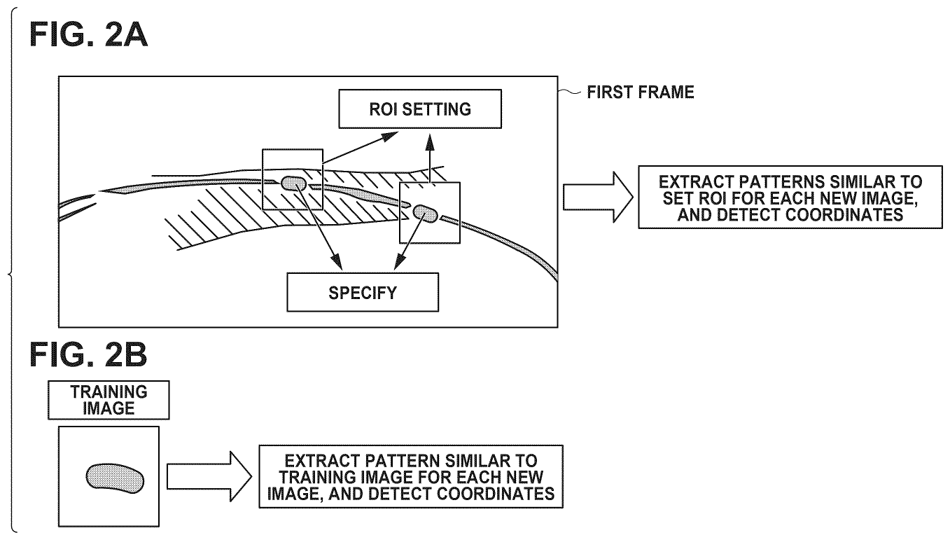

FIGS. 2A and 2B are diagrams illustrating processing of the detection function 212 according to an embodiment. For example, as illustrated in FIG. 2A, the display control function 215 described below controls the display 23 to display the X-ray image (first frame) that is first generated and stored in the storage circuitry 25. Referring to the first frame, the operator (for example, a doctor) specifies two stent markers in the first frame via the input circuitry 22, as illustrated in FIG. 2A. Thus, the detection function 212 recognizes the stent marker display pattern (such as shape and brightness information on the stent markers) in the X-ray image, and detects the coordinates of two stent markers in the first frame.

Subsequently, as illustrated in FIG. 2A, the detection function 212 sets a rectangle centered on each of the coordinates of the two stent markers specified in the first frame, as a Region of Interest (ROI). Then, by using a cross-correlation method, for example, the detection function 212 extracts patterns similar to the pattern within the set ROI for each sequentially generated new image, and detects the coordinates having the largest cross-correlation value as the coordinates of the stent markers.

While FIG. 2A illustrates a case in which the operator specifies stent markers at two positions, the present embodiment is not limited thereto. The operator may specify a stent marker at one position. In this case, the detection function 212 executes the cross-correlation method using the ROI set based on the coordinates of the specified stent marker also in the first frame, and detects the coordinates of the other stent marker in the first frame.

Alternatively, the detection function 212 detects the coordinates of the stent markers by using a training image that indicates the shape and brightness features of the stent markers attached to the stent actually used in the treatment in the X-ray images. For example, as illustrated in FIG. 2B, an X-ray image of the stent marker may be separately stored as a training image, the detection function 212 extracts a pattern similar to the training image, searches for the region having the highest similarity from the region candidates of the extracted stent markers, and detects the coordinates of the stent markers.

When detecting the coordinates of the stent markers from the sequentially generated X-ray images, the detection function 212 initially identifies the stent markers by using a plurality of X-ray images. More specifically, the detection function 212 identifies a plurality of target objects inserted into the body of the subject P and depicted in the X-ray images, by using a group of the sequentially generated X-ray images, and detects the coordinates of a plurality of target objects included in the newly generated X-ray images based on a result of the identification. For example, by using the stent markers specified by the operator or stent markers based on the training image, the detection function 212 extracts all regions similar to the stent markers for each of the plurality of X-ray images in a predetermined period. Then, the detection function 212 extracts the region most likely to be a stent marker, in a comprehensive way from among the regions extracted in the plurality of X-ray images, as the stent marker.

Hereinafter, the above-described processing for detecting and identifying stent markers is referred to as a “Learning mode”. In the stent marker detection in the Learning mode, for example, the detection function 212 detects a plurality of candidate target objects as a plurality of candidates of target objects included in the plurality of acquired X-ray images.

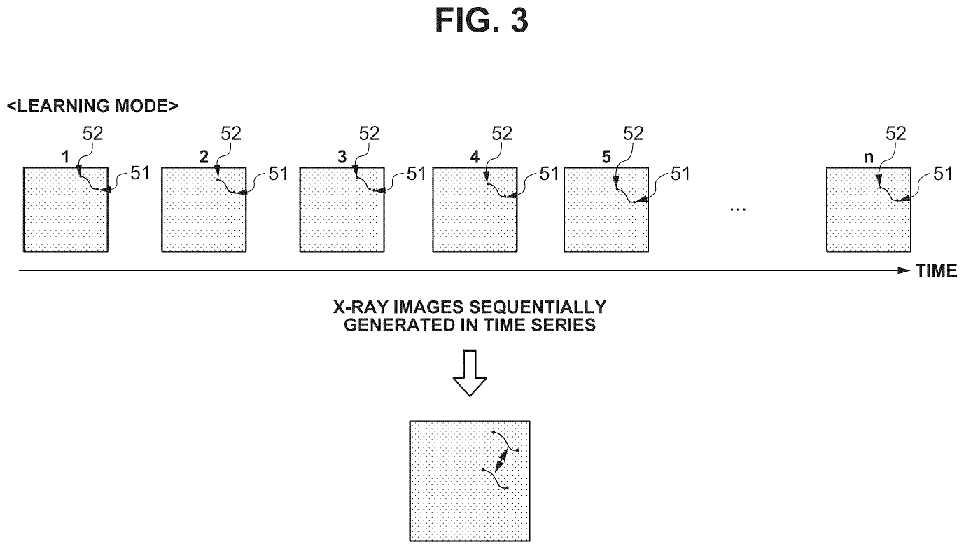

FIG. 3 is a diagram illustrating an example of the Learning mode according to an embodiment. FIG. 3 illustrates the Learning mode using n frames of X-ray images generated by the image processing circuitry 26. For example, the detection function 212 extracts all regions (coordinates) similar to stent markers from the entire area of the first frame illustrated in FIG. 3.

The processing circuitry 21 calculates an evaluation value for each of a plurality of candidate target objects based on the plurality of X-ray images by the evaluation function 213. For example, the evaluation function 213 forms pairs from all of the extracted coordinates and calculates an evaluation score for each pair based on the similarity and the distance between the coordinates. The evaluation function 213 calculates and assigns an evaluation score to a pair of coordinates 51 and 52. While FIG. 3 illustrates only the coordinates 51 and 52, if regions (coordinates) similar to stent markers are included, the coordinates of these regions are also detected, and pairs are formed with the coordinates 51, the coordinates 52 or other coordinates, and the evaluation scores are calculated.

For example, by using the evaluation score, the evaluation function 213 selects a pair candidate within the range of the stent length from among combinations of 10 marker candidates having the marker-likeness exceeding the threshold value. The evaluation function 213 performs tracking between frames for the selected pairs by using the center coordinates (gx, gy) of the selected pairs. For example, the evaluation function 213 generates a plurality of pair candidates (a plurality of candidate target objects) from candidate points of a plurality of markers for 100 frames (consecutive 100 X-ray images in time series). For example, pair candidates may be, for example, for 40 frames. The evaluation function 213 tracks each of the plurality of pairs (the plurality of candidate target objects) across multiple frames in time series, and generates a trajectory (track) of the pair in time series, and extends the trajectory across multiple frames. The evaluation function 213 calculates an evaluation value for the extended track for each of the plurality of pairs.

The evaluation function 213 calculates the evaluation value by using, for example, the distance between the ends of a pair connected across multiple frames in the track and a newly connected pair, the number of frames skipped during the pair connection related to the track generation, the frame number at which the track generation has started. In other words, the evaluation function 213 calculates the evaluation value by using at least one of the frequency of detection of a plurality of candidate target objects in a plurality of X-ray images and the temporal continuity in detection of a plurality of candidate target objects.

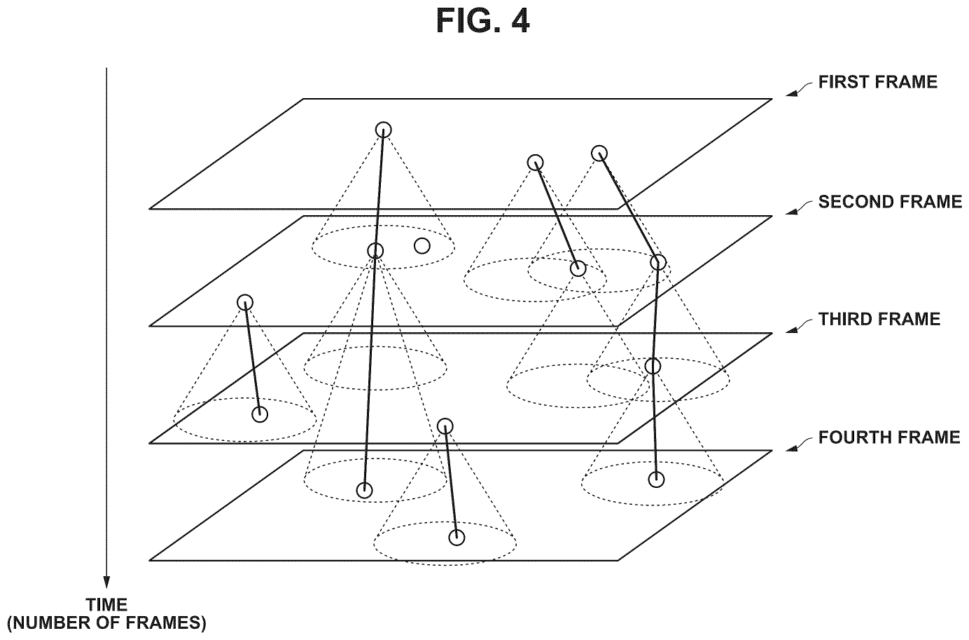

FIG. 4 is a diagram illustrating an example of tracking a plurality of candidate target objects across multiple frames. The circles illustrated in FIG. 4 correspond to the center coordinates of a plurality of selected candidate target objects. Straight lines connecting the circles between the frames indicate trajectories resulting from tracking of the pairs. As illustrated in FIG. 4, the evaluation function 213 tracks pairs, and generates and extends the trajectories. In this processing, the evaluation function 213 calculates the evaluation value for each of the trajectories such that there is no track interruption between frames, and the evaluation value increases as the trajectory becomes longer and remains uninterrupted between frames.

As described above, the evaluation function 213 calculates the evaluation value for each of a plurality of candidate target objects based on a plurality of X-ray images. The trajectory tracking and the evaluation value calculation are performed not only in the Learning mode but also during acquisition of X-ray image (in the Tracking Mode described below) by fluoroscopy of the subject P.

For example, since cardiac pulsation, lung expansion/contraction, and the like are cyclic (periodic), the stent markers that move in association with these exhibit cyclic (periodic) motion. In the above-described Learning mode, stent markers exhibiting cyclic (periodic) motion are comprehensively detected using X-ray images over a predetermined period, and the stent markers which are most likely to be stent markers are identified as the stent markers.

As described above, the detection function 212 first identifies stent markers in X-ray images in the Learning mode, and extracts regions encompassing possible coordinates of the stent markers. By detecting stent markers centered on the extracted regions, the detection function 212 can improve the detection accuracy.

The processing circuitry 21 generates a plurality of corrected images by performing correction processing on the plurality of X-ray images such that the position of a target object corresponding to a selected candidate target from among multiple object candidates and detected in a reference image among the multiple X-ray images is aligned with a reference position, via the corrected image generation function 214. By this correction processing, for example, the position of a target object coincides with the reference position in the corrected image. For example, the correction processing matches the position of the target object corresponding to the selected candidate target object with the reference position. Examples of reference images include an image in which the largest number of candidate target objects are detected in the Learning mode, and a Last Image Hold (LIH) image.

For example, the corrected image generation function 214 controls the image processing circuitry 26 to generate a corrected image by performing image transformation processing, such as affine transformation, on a new image so that the coordinates of the stent markers detected in the new image by the detection function 212 are aligned with reference coordinates corresponding to the stent markers previously detected by the detection function 212). The image transformation processing includes processing not involving scaling of the image, i.e., processing involving only image translation processing, such as parallel translation and rotation.

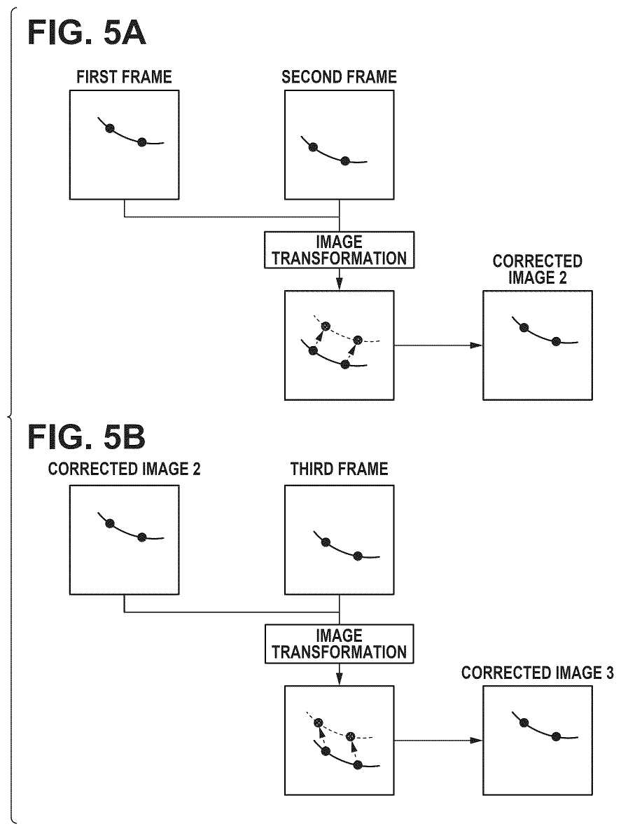

FIGS. 5A and 5B are diagrams illustrating processing of the corrected image generation function 214. FIGS. 5A and 5B illustrate processing that is applied to a new image in which the coordinates of the stent markers are detected based on a result of processing in the Learning mode executed by the detection function 212 after completion of the processing in the Learning mode. More specifically, the first frame illustrated in FIG. 5A indicates the X-ray image initially generated after the Learning mode has been completed.

For example, the detection function 212 first performs processing in the Learning mode by using images of 40 frames, and, as illustrated in FIG. 5A, detects the coordinates of the stent markers by using a result of the processing in the Learning mode for a first frame and a second frame generated after completion of the Learning mode. For example, the detection function 212 extracts regions similar to the stent markers centered on the regions extracted in the Learning mode, and detects, as stent markers, regions that are most likely to be stent markers among the extracted regions.

When the coordinates of the stent markers are detected by the detection function 212, as illustrated in FIG. 5A, the corrected image generation function 214 generates a corrected image 2 by image transformation from the second frame so that the coordinates of the stent markers detected in the X-ray image in the second frame generated as a new image are aligned with the coordinates (reference position) of the stent markers having already been detected in the first frame.

Then, for new images from a third frame onward, the corrected image generation function 214 generates a corrected image by using, as the reference coordinates, the coordinates of the stent markers in the corrected image generated by the corrected image generation function 214 from the X-ray image generated immediately preceding the new image. For example, as illustrated in FIG. 5B, the corrected image generation function 214 generates a corrected image 3 by the image transformation from the third frame so that the coordinates of the stent markers detected in the third frame are aligned with the coordinates of the stent markers in the corrected image 2 generated from the second frame.

In this embodiment, an example has been described in which the coordinates of the stent markers in the corrected image generated from the frame immediately preceding the new image are used as the reference coordinates. However, the present embodiment is not limited thereto. The coordinates of the stent markers detected in the first frame may be aligned with the reference coordinates and generate corrected images from new images of the second frame onward. Nevertheless, as described below, since the corrected images are used to generate display images to be used for moving image display, the corrected image may be generated from a new image by using the preceding corrected image.

As described above, the corrected image generation function 214 generates the corrected image in which the coordinates of the stent markers detected by the detection function 212 are aligned between images. More specifically, by using a result of processing in the Learning mode after stent markers have been identified in the Learning mode, the corrected image generation function 214 generates a corrected image by aligning, between images, the coordinates of the stent markers detected from subsequent X-ray images. The above-described process of generating corrected images will be referred to as the “Tracking mode”.

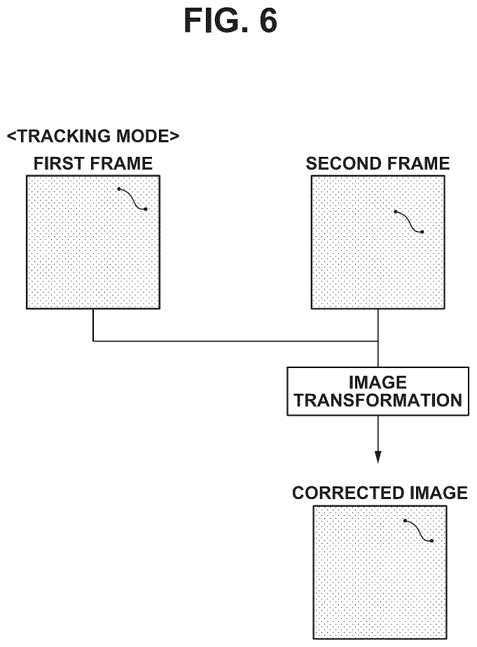

FIG. 6 is a diagram illustrating an example of a Tracking mode according to an embodiment. For example, in the Tracking mode, a corrected image is generated by transforming the image so that the coordinates of the stent markers detected centered on the regions extracted in the Learning mode are aligned, as illustrated in FIG. 6. More specifically, the corrected image generation function 214 generates a corrected image for the X-ray images in which stent markers have been detected by the detection function 212 after the Learning mode.

The display control function 215 causes the corrected images generated by the corrected image generation function 214 to be displayed as a moving image on the display 23. More specifically, each time a new corrected image is generated in time series, the display control function 215 performs control to sequentially display the newly generated corrected images as display images on the display 23. More specifically, the display control function 215 controls the display of the display images in which the coordinates of the stent markers are aligned. Accordingly, for example, although the background portion other than the stent appears blurred, the X-ray images can be displayed as a moving image in a state in which the stent portion remains stationary.

The display control function 215 controls the image processing circuitry 26 to display the display images generated by performing various filter processing on corrected images. For example, the display control function 215 controls the image processing circuitry 26 to perform high-frequency noise reduction filter processing using a recursive filter on the corrected images and to generate display images. The recursive filter is a filter for reducing high-frequency noise by adding pixel values of pixels of past frames, subjected to predetermined weighting, to pixel values of pixels of the frames being processed.

In corrected images, since the coordinates of the stent markers are aligned, even by using the recursive filter that utilizes past frames, it is possible to improve the visibility of the stent in corrected images by reducing high-frequency noise in the stent portion. The predetermined weighting in the recursive filter needs to be fixed at the time when the filter processing is executed, and may be determined before, during, and after the generation of corrected images.

More specifically, the display control function 215 generates display images with improved device visibility by sequentially executing recursive filter processing using past corrected images on sequentially generated corrected images, and displays the resulting images as a moving image. The display control function 215 can also generate display images by simply adding the sequentially generated corrected images.

As described above, the processing for displaying a moving image in which the device appears virtually stationary has been explained. The X-ray diagnostic apparatus 100 improves the image quality of the images displayed as a moving image in which the above-described device appears virtually stationary. As described above, when a moving image in which the device is virtually stationary is displayed, feature points (such as stent markers) of the device included in X-ray images are detected, and the positions of the detected feature points are approximately aligned, so that the movement of the device is virtually stopped.

In the above description, the set of markers at the center of X-ray images consists of two points ultimately selected. When a plurality of markers exists in X-ray images, there may be cases in which markers desired by the user are not selected. Further, a marker different from the one that the user intends to view may be displayed at the center of X-ray images, and the position of the device that the user originally wishes to view may be moved by pulsation or other movement.

Due to improved marker detection accuracy, a plurality of markers is more likely to be detected, resulting in cases in which a plurality of marker pairs having a high evaluation value exists. For example, balloons may be used in different ways during treatment or used in combination with a stent. Depending on the operating procedure, different balloons may be used, and therefore since a plurality of balloons is used, a plurality of balloons may be present within a blood vessel.

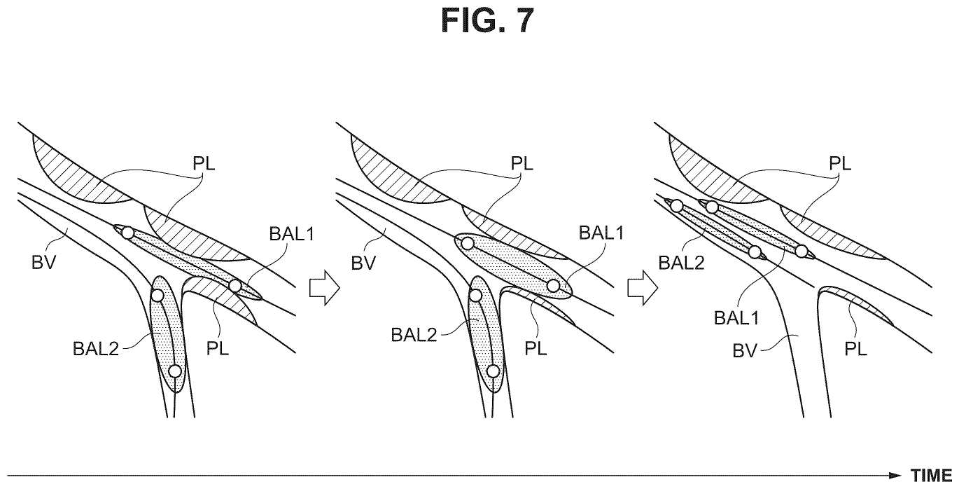

FIG. 7 is a diagram illustrating an example of using two balloons as a plurality of target objects. Circles illustrated in FIG. 7 indicate markers. In a blood vessel BV illustrated in FIG. 7, blood flows from left to right. As illustrated in FIG. 7, plaques PL are formed upstream and downstream of a bifurcation of the blood vessel BV. To prevent a part (thrombus) of the plaques PL from flowing downstream the bifurcation, a second balloon BAL2 is positioned and expanded downstream of the bifurcation of the blood vessel BV before the blood vessel BV is dilated at the plaques PL by a first balloon BAL1. Then, the first balloon BAL1 dilates the blood vessel BV at the plaques PL. Subsequently, the first balloon BAL1 and the second balloon BAL2 are positioned in the vicinity of the plaques PL in a larger blood vessel upstream of the bifurcation.

In the case as illustrated in FIG. 7, when the second balloon BAL2 is positioned downstream of the bifurcation of the blood vessel BV, the user wishes the second balloon BAL2 to be displayed as a target object at the center of the X-ray images. When the first balloon BAL1 is positioned at the position of the plaques PL after the dilation of the second balloon BAL2, the user wishes the first balloon BAL1 to be displayed as a target object at the center of the X-ray images. Further, in a case in which the first balloon BAL1 and the second balloon BAL2 are positioned in the vicinity of the plaques PL in the larger blood vessel upstream of the bifurcation, and when the user moves the first balloon BAL1, the user wishes the first balloon BAL1 to be displayed as a target object at the center of the X-ray images. When the user moves the second balloon BAL2, the user wishes the second balloon BAL2 to be displayed as a target object at the center of the X-ray images. As illustrated in FIG. 7, the user may wish to change the target object of interest during the procedure.

In the above description, when a trajectory (track) is generated during the marker detection, the user cannot select other trajectories (devices) not having been selected as virtually fixed display targets. Therefore, in the present embodiment, among a plurality of detected markers (a plurality of candidate target objects), marker pairs other than the trajectory having the highest evaluation value are stored in the storage circuitry 25. From among the stored marker pairs, selectable marker pairs are selected, and an operation is provided that allows the user to arbitrarily select a marker pair.

Specifically, marker pairs (a plurality of candidate target objects) having the marker-likeness exceeding the threshold value are stored in the storage circuitry 25 as a Track list together with the value of the marker-likeness, evaluation value, coordinates, and the like. In this processing, a flag is assigned to the candidate target object having the highest evaluation value in the Track list. Furthermore, in the Track list, identifiers for distinguishing each of the plurality of candidate target objects are assigned in the Track list. These identifiers can be arbitrarily set to, a name, number, abbreviation, and the like, according to a user instruction input via the input circuitry 22.

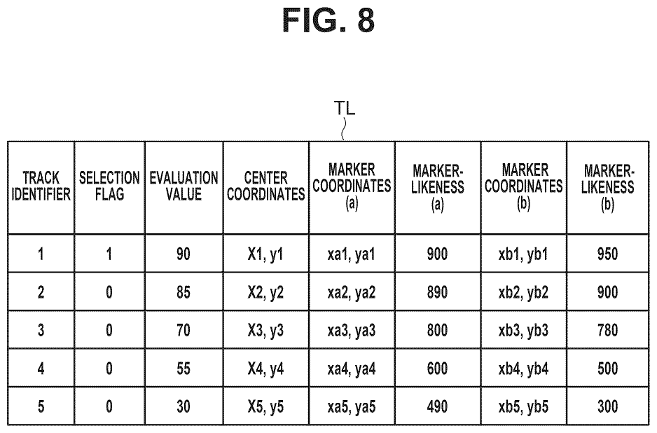

FIG. 8 is a diagram illustrating an example of a Track list TL. For example, upon completion of the Learning mode, the Track list TL is generated and stored in the storage circuitry 25 by the evaluation function 213, for example. As illustrated in FIG. 8, the Track list TL indicates a plurality of target objects sorted in order of the evaluation value.

By the display control function 215, the processing circuitry 21 arranges the candidate target objects based on the evaluation value of the track and displays the candidate target objects on the display 23. When the user wishes to view a different device during the procedure or move a balloon for different use during the procedure, as illustrated in FIG. 7, the user can easily change the markers to be displayed at the center of the X-ray images by selecting a desired target candidate through an operation via the input circuitry 22. The operation for selecting a candidate target object can be implemented, for example, on a fluoroscopic image, a captured image, an LIH display, an operation screen in the Tracking mode, and a collected moving and still image on the tablet terminal.

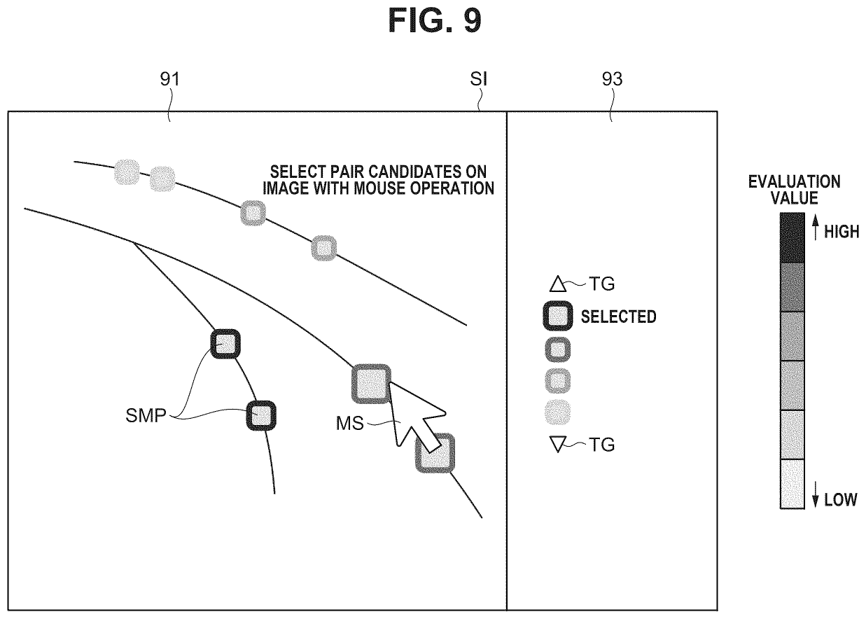

FIG. 9 is a diagram illustrating an example of a marker selection screen SI corresponding to candidate target objects. The marker selection screen SI includes an X-ray image 91 and a marker list (marker list display) 93. As the X-ray image 91 in the marker selection screen SI, for example, the reference image is used. As illustrated in FIG. 9, a marker pair SMP currently selected is displayed in highlighted manner on the display 23. The highlight may be represented, for example, by a line types, such as a solid line or a dotted line, a color tone, such as dark or light color, a change in size, and a frame.

More specifically, the display 23 displays the selected candidate target objects from among the plurality of candidate target objects in highlighted manner. As illustrated in FIG. 9, selectable marker pairs are displayed in order of the evaluation value in the marker list 93 in the selection screen SI. In this state, when the user moves a selection target object by operating a toggle TG button in the marker list 93, the target object is determined. Furthermore, when a mouse cursor MS comes into contact with a marker (candidate target object) in the marker list 93, the display control function 215 causes the contacted marker to be displayed in highlighted manner, for example, by enlarging or blinking.

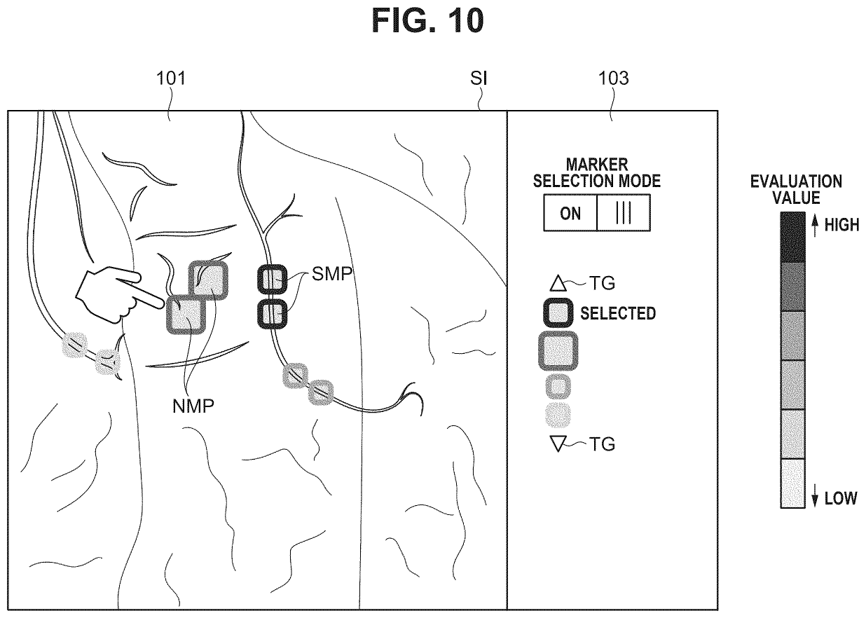

FIG. 10 is a diagram illustrating an example of a selection screen SI displayed on the tablet terminal. The marker selection screen SI includes an X-ray image 101 and a marker list 103. As illustrated in FIG. 10, when the user turns ON a marker selection mode in the marker list 103, the display control function 215 displays the currently selected pair and selectable pair candidates in different colors. More specifically, as illustrated in FIG. 10, the currently selected marker pair (candidate target objects) SMP and selectable marker pairs are displayed in highlighted manner.

The display form illustrated in FIG. 10 is similar to that illustrated in FIG. 9. FIG. 10 illustrates a state in which, when the user performs a touch or mouse operation at a position in the vicinity of a marker pair, the display control function 215 displays a marker pair NMP in the vicinity of the position of the operation in highlighted manner. As illustrated in FIG. 10, the marker list 103 displays candidate target objects in a list form in order of the evaluation value, allowing a toggle selection by a button operation via a toggle TG. In this processing, the display form of candidate target objects in the toggle selection is linked to the selection state in the X-ray image 101 and the marker list 103.

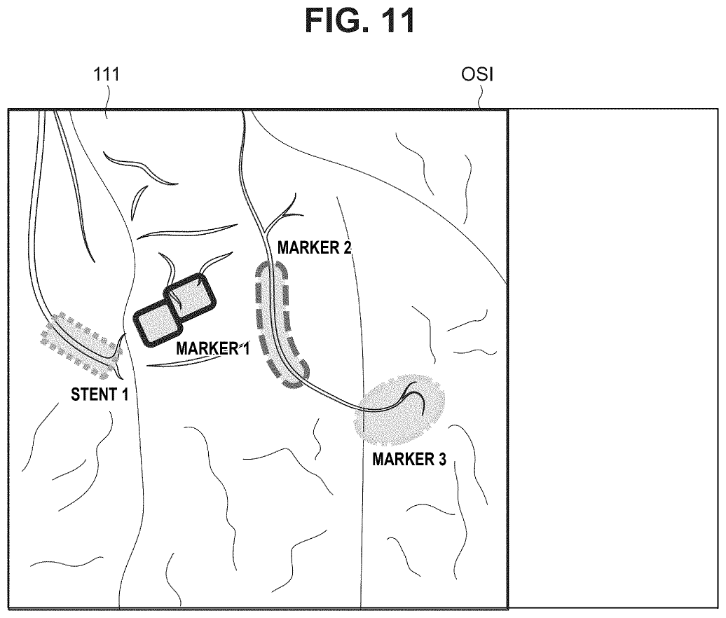

FIG. 11 is a diagram illustrating an example of another selection screen OSI. The other selection screen OSI includes an X-ray image 111 indicating the regions of markers and a treatment device (a stent 1 in FIG. 11). As illustrated in FIG. 11, neighboring areas of the plurality of candidate target objects are displayed in highlighted manner by closed curves, rectangles, or the like, using line types or color tones corresponding to the evaluation value. In this processing, as illustrated in FIG. 11, identifiers for identifying marker pairs may be displayed in the vicinity of the neighboring areas. A candidate target object is selected by selecting the neighboring area. The display control function 215 may display the above-described neighboring areas when the evaluation value and/or the value of the marker-likeness are small. As illustrated in FIG. 11, the display 23 may display identifiers (such as names and numbers) that allow a plurality of candidate target objects to be identified.

A plurality of candidate target objects displayed on the selection screen SI and the other selection screen OSI, i.e., a set of marker candidates is not limited to two points. For example, when a set of marker candidates includes three or more points, the center point based on all the markers is tracked. More specifically, in an example case where a device such as a stent and a balloon is provided with four different markers, the detection function 212 calculates the center point (such as the center of gravity) of coordinates of the four points of the markers, and tracks the device by using the center point.

When a track that has been continuously maintained does not appear in the latest frame (LIH image), the display control function 215 may display a plurality of candidate target objects appearing in frames retrieved from previous frames of the LIH image, in the selection screen SI and the other selection screen OSI. To search the previous frames, for example, the display control function 215 may use the X-ray images corresponding to the end-diastole acquired in electrocardiographic synchronization.

For example, when the timing of selection of the markers corresponding to a candidate target object is the LIH image, the tracking mode is immediately reflected in the next fluoroscopic imaging. More specifically, as illustrated in FIG. 10, without turning ON the marker selection mode, the Tracking mode is performed for selected markers according to the marker selection. Even during fluoroscopic imaging or image capturing, it is possible to accept a candidate target object. In this processing, markers at positions at other than the center position of the X-ray images are highly likely to be moving due to pulsation of the subject P. However, as illustrated in FIGS. 9 and 10, the user can select markers by operating a toggle TG.

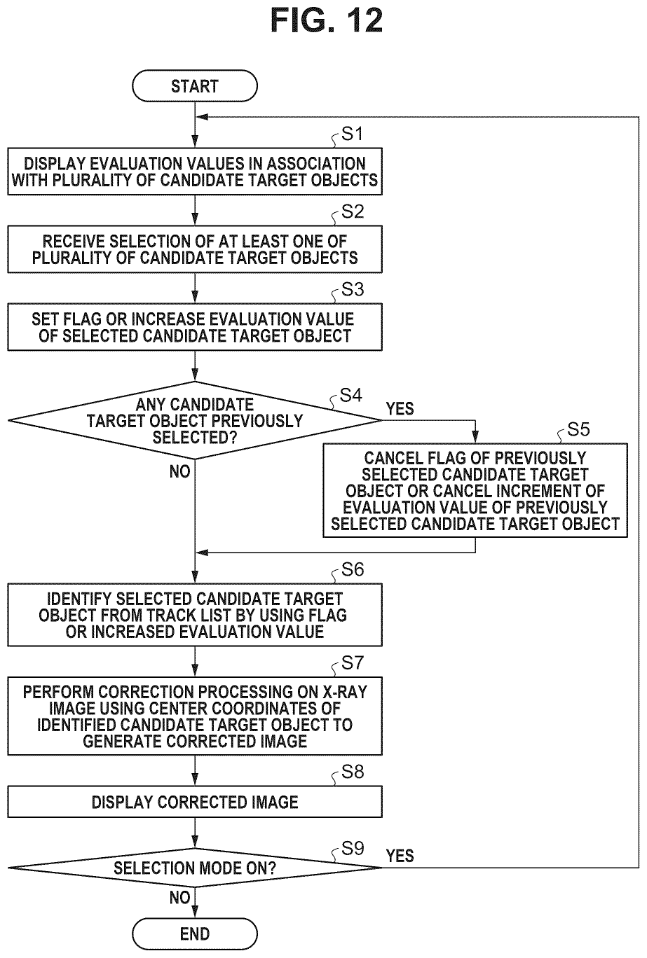

The configuration of the X-ray diagnostic apparatus 100 according to an embodiment has been described above. Hereinafter, processing for selecting one of a plurality of candidate target objects and executing the Tracking mode (hereinafter referred to as target object selection Tracking processing) will be described below with reference to FIG. 12.

FIG. 12 is a flowchart illustrating an example of a procedure of the target object selection Tracking processing. The following describes a case in which the Learning mode has been executed prior to the target object selection Tracking processing. A plurality of candidate target objects has reliability (marker-likeness) exceeding the threshold value, and has been stored in the storage circuitry 25 as Track list TL, as illustrated in FIG. 8. As illustrated in FIG. 8, the Track list TL includes a plurality of tracks that have the evaluation value equal to or larger than a predetermined value and are sorted in order of their evaluation values.

### Target Object Selection Tracking Processing

### Step S1

An operation for turning ON the marker selection mode is input via the input circuitry 22. In response to the operation, the display control function 215 causes the display 23 to display the evaluation values in association with a plurality of candidate target objects in the marker lists 93 and 103, as illustrated in FIGS. 9 and 10. For example, the display control function 215 causes the display 23 to display the selection screen SI. The display 23 displays the evaluation values in association with the plurality of candidate target objects in the marker lists 93 and 103, as illustrated in FIG. 9. In this processing, the display 23 further displays the plurality of candidate target objects in the reference images (X-ray images 91 and 101). As illustrated in FIGS. 9 and 10, the display 23 may display the plurality of candidate target objects in a list form in the marker lists 93 and 103. The display 23 may further display a plurality of identifiers for enabling identification of the plurality of candidate target objects in the X-ray image 111, as illustrated in FIG. 11.

### Step S2

By an operation by the user via the input circuitry 22, a selection of at least one of the plurality of candidate target objects is accepted. More specifically, the input circuitry 22 accepts a selection of at least one of the plurality of candidate target objects. For example, the input circuitry 22 accepts a selection of at least one of the plurality of candidate target objects displayed in the reference image. In this processing, the display 23 displays the selected candidate target object from among the plurality of candidate target objects in the highlighted manner.

The input circuitry 22 may accept a selection of at least one of the plurality of candidate target objects in response to a button operation performed on the toggle TG indicating the selection of the plurality of candidate target objects in the list displayed in the selection screen SI. When a plurality of identifiers for enabling identification of a plurality of candidate target objects is displayed in the X-ray image 111, as illustrated in FIG. 11, the input circuitry 22 may accept a selection of at least one of the plurality of identifiers. In a case where the input circuitry 22 can accept a selection of a candidate target object in the other selection screen OSI as illustrated in FIG. 11, the processing in step S1 becomes unnecessary.

### Step S3

The processing circuitry 21 assigns a flag to or increases the evaluation value with respect to the selected candidate target object via the evaluation function 213. For example, the evaluation function 213 assigns a flag to the selected candidate target object in the Track list TL. More specifically, the evaluation function 213 changes the “Selection Flag” for the selected candidate target object in the Track list TL from 0 to 1. Subsequently, the evaluation function 213 updates the Track list TL and then stores the list in the storage circuitry 25.

Alternatively, the evaluation function 213 recalculates the evaluation value of the selected candidate target object. For example, the evaluation function 213 recalculates the evaluation value of the selected candidate target object by using the neighboring value from the candidate target object having the highest evaluation value and/or the marker-likeness (Trustness) used at the time of marker candidate point selection for the last (latest) frame (for example, the LIH image). In this processing, the evaluation value can be temporarily increased to set an evaluation state exceeding the evaluation of the candidate target object having the highest evaluation value, and continue the tracking in that state. The evaluation function 213 changes the evaluation value before the recalculation to the recalculated evaluation value in the Track list TL. Subsequently, the evaluation function 213 updates the Track list TL and then stores the list in the storage circuitry 25.

In a case where the neighboring value from the candidate target object having the highest evaluation value, for example, the evaluation function 213 recalculates the evaluation value by using the following Equation (1):

Re-evaluation value=Evaluation value+α×(1−(Distance from candidate target object having highest evaluation value/Maximum distance on-screen)) (1)

The constant α in Equation (1) represents a weighting factor and can be appropriately adjusted (variable) according to conditions, such as the image quality.

In a case where assigning the marker-likeness, the evaluation function 213 recalculates the evaluation value by using the following Equation (2):

Re-evaluation value=Evaluation value+β×Marker-likeness in last frame (LIH image) (2)

The constant β in Equation (2) represents a weighting factor and can be appropriately adjusted (variable) according to conditions, such as the image quality.

In a case where the evaluation value of the selected candidate target object is to be increased, the evaluation function 213 recalculates the evaluation value, for example, by using the following Equation (3):

Re-evaluation value=Evaluation value+Increment value (3)

The increment value in Equation (3) is set to a value larger than (Maximum evaluation value-Minimum evaluation value) in the Track list TL. In a case where the evaluation value has already been recalculated, the increment value in Equation (3) is set to a value larger than (Maximum re-evaluation value-Minimum re-evaluation value).

### Step S4

In a case where there is any previously selected candidate target object (YES in step S4), i.e., in a case where any candidate target object has been previously selected in the Track list TL, the processing proceeds to step S5. In a case where there is no previously selected candidate target object (NO in step S4), i.e., in a case where no candidate target object has been previously selected in the Track list TL, the processing proceeds to step S6.

### Step S5

The processing circuitry 21 removes the flag of the previously selected candidate target object or cancels the increment of the previously selected candidate target object via the evaluation function 213. More specifically, the evaluation function 213 changes the “Selection Flag” of the previously selected candidate target object in the Track list TL from 1 to 0. The previously selected candidate target object is different from the candidate target object selected in step S2. The evaluation function 213 changes the evaluation value of the previously selected candidate target object to the evaluation value before the increment in the Track list TL. Subsequently, the evaluation function 213 updates the Track list TL and then stores the list in the storage circuitry 25.

### Step S6

The processing circuitry 21 identifies the selected candidate target object from the updated Track list by using the flag or the increased evaluation value, via the evaluation function 213. When using the flag, the evaluation function 213 identifies the candidate target object whose selection flag is “1” in the updated Track list. When using the increased evaluation value, the evaluation function 213 identifies the candidate target object having the highest evaluation value in the updated Track list.

### Step S7

The processing circuitry 21 performs the correction processing on the X-ray images by using the center coordinates of the identified candidate target object via the corrected image generation function 214. As a result, the corrected image generation function 214 generates a corrected image. For example, when fluoroscopic imaging is performed on the subject P, a plurality of corrected images is generated until the fluoroscopic imaging is completed.

### Step S8

The processing circuitry 21 causes the display 23 to display the generated corrected image via the display control function 215. Thus, the display 23 displays the corrected image.

### Step S9

In a case where the marker selection mode is turned ON by a user instruction via the input circuitry 22 (YES in step S9), the processing returns to step S1. Then, the processing from step S1 is repeated. In a case where the marker selection mode is not turned ON (NO in step S9), the processing circuitry 21 ends the target object selection Tracking processing. In a case where the selection of the candidate target object is accepted without activation of the marker selection mode, for example, through a button operation of a toggle TG, the processing in step S3 and subsequent steps is repeated.

The above-described X-ray diagnostic apparatus 100 according to an embodiment performs acquiring sequentially a plurality of X-ray images in time series, detecting a plurality of candidate target objects as candidates of a plurality of target objects included in the plurality of acquired X-ray images, calculating evaluation values each for a different candidate target object of the plurality of candidate target objects based on the plurality of X-ray images, displaying the calculated evaluation values each in association with the corresponding candidate target object of the plurality of candidate target objects, and accepting a selection of at least one of the plurality of candidate target objects, and generates a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions. Thus, the X-ray diagnostic apparatus 100 according to an embodiment can virtually fix and display the candidate target object intentionally selected by the user at the center of the X-ray images. Therefore, the X-ray diagnostic apparatus 100 according to an embodiment can improve the visibility of the selected target object in the X-ray images.

The X-ray diagnostic apparatus 100 according to an embodiment calculates the evaluation value by using at least one of the frequency of the detection of a plurality of candidate target objects in a plurality of X-ray images and the temporal continuity of the detection. Thus, the X-ray diagnostic apparatus 100 according to an embodiment can efficiently evaluate a plurality of candidate target objects desired by the user.

In the X-ray diagnostic apparatus 100 according to an embodiment, the target object is at least one of a treatment device inserted into the body of the subject P and a feature object attached to the treatment device. In the X-ray diagnostic apparatus 100 according to an embodiment, the treatment device includes a plurality of feature objects. The X-ray diagnostic apparatus 100 according to an embodiment can virtually fix and display the treatment device intentionally selected by the user or the feature object attached to the treatment device at the center of the X-ray images, whereby the visibility of the treatment device selected in the X-ray images or the feature object attached to the therapeutic device is improved.

The X-ray diagnostic apparatus 100 according to an embodiment highlights the selected candidate target object among the plurality of candidate target objects. Thus, the X-ray diagnostic apparatus 100 according to an embodiment enables the user to easily recognize the candidate target object selected by the user.

The X-ray diagnostic apparatus 100 according to an embodiment further displays a plurality of candidate target objects in the reference image, and accepts a selection of at least one of the plurality of candidate target objects displayed in the reference image. Thus, the X-ray diagnostic apparatus 100 according to an embodiment enables the user to easily select a desired candidate target object from the plurality of candidate target objects.

The X-ray diagnostic apparatus 100 according to an embodiment displays a plurality of candidate target objects in a list and accepts a selection of at least one of the plurality of candidate target objects in response to a button operation of the toggle TG indicating a selection of a plurality of candidate target objects in the list. Therefore, the X-ray diagnostic apparatus 100 according to an embodiment enables the user to select a desired candidate target object at any desired timing through the button operation of the toggle TG.

The X-ray diagnostic apparatus 100 according to an embodiment further displays a plurality of identifiers for enabling identification of a plurality of candidate target objects, and accepts a selection of at least one of the plurality of identifiers. Therefore, the X-ray diagnostic apparatus 100 according to an embodiment enables the user to select a desired candidate target object by selecting a preset identifier.

When performing treatment by using a plurality of balloons or stents in the cardiovascular region, for example, the X-ray diagnostic apparatus 100 according to an embodiment enables the user to select a desired marker position according to the treatment process or procedure. Therefore, the X-ray diagnostic apparatus 100 according to an embodiment can enhance the effect of displaying the device selected by the user as virtually fixed at the center of the image. Even in a case where the marker of the target object related to fixed display is out of the imaging visual field, the X-ray diagnostic apparatus 100 according to an embodiment allows the user to select other marker pairs around the target object, and by continuously performing the Tracking mode, and thus the risk of interrupting the procedure can be reduced.

When the technical concept according to the present embodiment is implemented in a medical image processing apparatus, the medical image processing apparatus includes an acquisition unit configured to sequentially acquire a plurality of X-ray images in time series, a detection unit configured to detect a plurality of candidate target objects as candidates of a plurality of target objects included in the plurality of acquired X-ray images, an evaluation unit configured to calculate evaluation values each for a different candidate target object of the plurality of candidate target objects based on the plurality of X-ray images, a display unit configured to display the evaluation values each in association with the corresponding candidate target object of the plurality of candidate target objects, an acceptance unit configured to accept a selection of at least one of the plurality of candidate target objects, and a corrected image generation unit configured to generate a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.

The medical image processing apparatus can be implemented in various servers or an information processing apparatus on a network. The medical image processing apparatus may be mounted on the X-ray diagnostic apparatus. The acquisition unit of the medical image processing apparatus is mounted as an acquisition function on the processing circuitry. In this case, the acquisition function sequentially acquires a plurality of X-ray images in time series from the X-ray diagnostic apparatus. The acquisition function stores the sequentially acquired X-ray images in the storage circuitry. The hardware configurations, functions, and processing of the detection unit, the evaluation unit, the display unit, the acceptance unit, and the corrected image generation unit in the medical image processing apparatus conform to an embodiment, and the redundant descriptions will be omitted. Procedures and effects of the target object selection Tracking processing implemented by the medical image processing apparatus are similar to an embodiment, and the redundant descriptions will be omitted.

When the technical concept according to the present embodiment is implemented in a medical image processing method, the medical image processing method includes acquiring sequentially a plurality of X-ray images in time series, detecting a plurality of candidate target objects as candidates of a plurality of target objects included in the plurality of acquired X-ray images, calculating evaluation values each for a different candidate target object of the plurality of candidate target objects based on the plurality of X-ray images, displaying the evaluation values each in association with the corresponding candidate target object of the plurality of candidate target objects, accepting a selection of at least one of the plurality of candidate target objects, and generating a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions. Procedures and effects of the target object selection Tracking processing implemented by the medical image processing method are similar to those according to an embodiment, and the redundant descriptions will be omitted.

When the technical concept according to the present embodiment is implemented by a medical image processing program, the medical image processing program causes a computer to implement acquiring sequentially a plurality of X-ray images in time series, detecting a plurality of candidate target objects as candidates of a plurality of target objects included in the plurality of acquired X-ray images, calculating evaluation values each for a different candidate target object of the plurality of candidate target objects based on the plurality of X-ray images, displaying the evaluation values each in association with the corresponding candidate target object of the plurality of candidate target objects, accepting a selection of at least one of the plurality of candidate target objects, and generating a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.

For example, the target object selection Tracking processing can be implemented by installing a medical image processing program in the computer and then loading the medical image processing program in a memory. In this case, a program for causing the computer to execute the target object selection Tracking process can be distributed being stored in a storage medium including a magnetic disk (such as a hard disk), an optical disk (such as a compact disc read only memory (CD-ROM) and a digital versatile disc (DVD)), and a semiconductor memory. For example, the distribution of the medical image processing program is not limited to the above-described medium. The program may be distributed by using an electrical communication function, such as downloading via the Internet. Procedures and effects of the target object selection Tracking processing implemented by the medical image processing program are similar to those according to an embodiment, and the redundant descriptions will be omitted.

According to the above-described embodiment, visibility of selected target objects in X-ray images is improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
image processing circuitry configured to sequentially acquire a plurality of X-ray images in time series;
processing circuitry configured to
detect a plurality of candidate target objects as candidates of a plurality of target objects included in the plurality of acquired X-ray images, and
calculate evaluation values each for a different candidate target object of the plurality of candidate target objects based on the plurality of X-ray images;
a display configured to display the evaluation values each in association with the corresponding candidate target object of the plurality of candidate target objects; and
input circuitry configured to accept a selection of at least one of the plurality of candidate target objects,
wherein the processing circuitry generates a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.

2. The X-ray diagnostic apparatus according to claim 1, wherein the processing circuitry calculates the evaluation values by using at least one of a frequency of detection of the plurality of candidate target objects in the plurality of X-ray images and a temporal continuity of the detection.

3. The X-ray diagnostic apparatus according to claim 1, wherein the target object is at least one of a treatment device inserted into a subject body and a feature object attached to the treatment device.

4. The X-ray diagnostic apparatus according to claim 3, wherein the treatment device is provided with a plurality of the feature objects.

5. The X-ray diagnostic apparatus according to claim 1, wherein the display highlights the selected candidate target object among the plurality of candidate target objects.

6. The X-ray diagnostic apparatus according to claim 1,
wherein the display further displays the plurality of candidate target objects in the reference image, and
wherein the input circuitry accepts a selection of at least one of the plurality of candidate target objects displayed in the reference image.

7. The X-ray diagnostic apparatus according to claim 1,
wherein the display displays the plurality of candidate target objects in a list, and
wherein the input circuitry accepts a selection of at least one of the plurality of candidate target objects in response to a toggle button operation indicating a selection of the plurality of candidate target objects in the list.

8. The X-ray diagnostic apparatus according to claim 1,
wherein the display further displays a plurality of identifiers for enabling identification of the plurality of candidate target objects, and
wherein the input circuitry accepts a selection of at least one of the plurality of identifiers.

9. A medical image processing apparatus comprising:
image processing circuitry configured to sequentially acquire a plurality of X-ray images in time series;
processing circuitry configured to
detect a plurality of candidate target objects as candidates of a plurality of target objects included in the plurality of acquired X-ray images, and
calculate evaluation values each for a different candidate target object of the plurality of candidate target objects based on the plurality of X-ray images;
a display configured to display the evaluation values each in association with the corresponding candidate target object of the plurality of candidate target objects; and
input circuitry configured to accept a selection of at least one of the plurality of candidate target objects,
wherein the processing circuitry generates a plurality of corrected images by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.

10. A medical image processing method comprising:
acquiring sequentially a plurality of X-ray images in time series;
detecting a plurality of candidate target objects as candidates of a plurality of target objects included in the plurality of acquired X-ray images;
calculating evaluation values each for a different candidate target object of the plurality of candidate target objects based on the plurality of X-ray images;
displaying the evaluation values each in association with the corresponding candidate target object of the plurality of candidate target objects; and
accepting a selection of at least one of the plurality of candidate target objects,
wherein a plurality of corrected images is generated by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.

11. A computer-readable non-transitory storage medium for storing a medical image processing program for causing a computer to implement:
acquiring sequentially a plurality of X-ray images in time series;
detecting a plurality of candidate target objects as candidates of a plurality of target objects included in the plurality of acquired X-ray images;
calculating evaluation values each for a different candidate target object of the plurality of candidate target objects based on the plurality of X-ray images;
displaying the evaluation values each in association with the corresponding candidate target object of the plurality of candidate target objects; and
accepting a selection of at least one of the plurality of candidate target objects,
wherein a plurality of corrected images is generated by performing, on the plurality of X-ray images, correction processing in which positions of the target objects that have been detected in a reference image among the plurality of X-ray images and correspond to the candidate target object selected from among the plurality of candidate target objects are set as reference positions, and positions of the target objects corresponding to the selected candidate target object are aligned with the reference positions.
